(19) United States
(12) Patent Application Publication
(10) Pub. No.: US 2010/0174343 A1
(43) Pub. Date: Jul. 8, 2010

(54) APPARATUS AND METHODS FOR FACILITATING WOUND HEALING AND TREATING SKIN

(75) Inventors: Rafael Victor Andino, Lawrenceville, GA (US); Christopher J. Brooks, Glen Head, NY (US); Michael J. Keating, Hardwick, NJ (US); Courtney F. Morgan, Alpharetta, GA (US); Donald Van Royen, New York, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: Biofisica LLC, Atlanta, GA (US)

(21) Appl. No.: 12/537,670

(22) Filed: Aug. 7, 2009

Related U.S. Application Data
(63) Continuation of application No. 11/494,819, filed on Jul. 28, 2006, now abandoned.

(51) Int. Cl. A61N 1/02

(52) U.S. Cl. 607/50

(57) ABSTRACT

Electrode systems and methods are provided for applying electrical stimulation to a wound or skin. The electrode systems may include a feedback sensor configured to detect at least one wound healing factor or other treatment factors and may adjust the electrical stimulation based on feedback sensor measurements. The electrode systems may include multiple center electrodes for attachment to the wound. Multiple therapies may be stored on the electrode systems and a selected one of the stored therapies may be applied. A timer may be included to indicate the length of time a therapy has been applied. The electrode system may include a control module configured as a flexible circuit which conforms to the shape of the wound or the skin to which it is applied. In addition, a medical kit may be supplied for use in applying therapies, including multiple control modules and multiple electrodes.

The present invention relates generally to apparatus and methods for facilitating wound healing and treating skin through the use of electrical stimulation.

### BACKGROUND OF THE INVENTION

Connective tissue wound healing typically occurs in three distinct phases. Although these phases intertwine and overlap, each has a specific sequence of events that distinguishes it. During the initial, or inflammatory phase, the body begins to clean away bacteria and initiate hemostasis. The inflammatory phase has three subphases: hemostasis; leukocyte and macrophage migration; and epithelialization. This phase typically lasts for about four days.

The second phase, the proliferative phase, is characterized by a proliferation of fibroblasts, collagen synthesis, granulation, and wound contraction. The proliferative phase typically begins about 48 hours after the wound is inflicted and can extend anywhere from two hours up to a week. In this phase, the fibroblast cells begin the synthesis and deposition of the protein collagen, which will form the main structural matrix for the successful healing of the wound.

In the third phase, the remodeling phase, the collagen production slows. The collagen that is formed in this stage is more highly organized than the collagen formed in the proliferative phase. Eventually, the remodeled collagen increases the tensile strength in the wound and returns the wound to about 80% of the skin's original strength.

This is the general process that occurs in healthy human beings. Patients that suffer from conditions which limit the flow of blood to the wound site are unfortunately not able to exhibit the normal wound healing process as described. In some patients this process can be halted. Factors that can negatively affect this normal wound healing process include diabetes, impaired circulation, infection, malnutrition, medication, and reduced mobility. Other factors such as traumatic injuries and burns can also impair the natural wound healing process.

Poor circulation, for varying reasons, is the primary cause of chronic wounds such as venous stasis ulcers, diabetic ulcers, and decubitus foot ulcers. Venous stasis ulcers typically form just above the patient's ankles. The blood flow in this region of the legs in elderly or incapacitated patients can be very sluggish, leading to drying skin cells. These skin cells are thus oxygen starved and poisoned by their own waste products and begin to die. As they do so, they leave behind an open leg wound with an extremely poor chance of healing on its own. Diabetic foot ulcers form below the ankle, in regions of the foot that have very low levels of circulation.

Similarly, decubitus ulcers form when skin is subjected to constant compressive force without movement to allow for blood flow. The lack of blood flow leads to the same degenerative process as described above. Paraplegics and severely immobile elderly patients who lack the ability to toss and turn while in bed are the main candidates for this problem.

Traditional approaches to the care and management of these types of chronic non-healing wounds have included passive techniques that attempt to increase the rate of repair and decrease the rate of tissue destruction. Examples of these techniques include antibiotics, protective wound dressings, removal of mechanical stresses from the affected areas, and the use of various debridement techniques or agents to remove wound exudate and necrotic tissue.

For the most part, these treatment approaches are not very successful. The ulcers can take many months to heal and in some cases they may never heal or they may partially heal only to recur at some later time.

Active approaches have been employed to decrease the healing time and increase the healing rates of these ulcers. These approaches may include surgical treatment as well as alterations to the wound environment. These alterations may include the application of a skin substitute impregnated with specific growth factors or other agents, the use of hyperbaric oxygen treatments, or the use of electrical stimulation. It has also been shown experimentally (both in animal and clinical trials) that specific types of electrical stimulation will alter the wound environment in a positive way so that the normal wound healing process can occur or in some cases occur in an accelerated fashion.

The relationship between direct current electricity and cellular mitosis and cellular growth has become better understood during the latter half of the twentieth century. Weiss, in Weiss, Daryl S., et. al., Electrical Stimulation and Wound Healing, Arch Dermatology, 126:222 (February 1990), points out that living tissues naturally possess direct current electropotentials that regulate, at least in part, the wound healing process. Following tissue damage, a current of injury is generated that is thought to trigger biological repair. This current of injury has been extensively documented in scientific studies. It is believed that this current of injury is instrumental in ensuring that the necessary cells are drawn to the wound location at the appropriate times during the various stages of wound healing. Localized exposure to low levels of electrical current that mimic this naturally occurring current of injury has been shown to enhance the healing of soft tissue wounds in both human subjects and animals. It is thought that these externally applied fields enhance, augment, or take the place of the naturally occurring biological field in the wound environment, thus fostering the wound healing process.

U.S. Pat. No. 6,631,294 to Andino discloses an electrode system that generates a current flow that envelops and permeates a wound site. The system includes two electrodes, adapted and positioned to cause a current to flow from one electrode through the wound to the other electrode. However, this system does not include a sensor to monitor the wound or control the current flow based on sensor measurements.

In view of the foregoing, it is an object of the present invention to provide improved systems and methods for applying electrical stimulation to wounds and skin.

It is a more particular object of the present invention to monitor wound healing parameters or indicators of wound healing, and skin treatment factors using one or more sensors.

It is also an object of the present invention to adjust an applied therapy based on feedback from one or more sensors.

It is also an object of the present invention to store multiple therapies with an electrode system.

It is also an object of the present invention to provide components of electrode systems that selectively couple to other components.

It is also an object of the present invention to provide flexible components to conform to the shape of the area to which the electrode system is applied.

It is also an object of the present invention to provide electrode systems with more than two electrodes to more evenly distribute the electrical stimulation.

It is also an object of the present invention to provide control modules of different strengths and electrodes of different sizes, shapes, and configurations.

### SUMMARY OF THE INVENTION

These and other objects of the invention are accomplished in accordance with the principles of the present invention by providing an electrode system for applying a therapy to a wound or skin that includes a feedback sensor. The electrode system may include a control module and first and second electrodes. The first electrode may be applied to the wound, while the second electrode may be applied to skin surrounding the wound. The control module is configured to apply a voltage potential across the first electrode and the second electrode. The feedback sensor is coupled to the control module, and is configured to detect at least one factor that affects wound growth or a treatment factor. The feedback sensor provides an output to the control module, and the control module may adjust the voltage potential based on the output from the feedback sensor. By adjusting the voltage potential, the control module adjusts the therapy applied to the wound.

The feedback sensor may be configured to detect any suitable wound healing parameter to include a biological factor such as a growth factor or factors or other treatment factors. According to one arrangement, the wound healing factor (such as a growth factor or other parameter in the wound healing process) detected by the feedback sensor includes one or more of: a natural current of injury of the wound, an amount of peroxide being generated by the first electrode or present within the wound bed, a temperature of the wound, and a temperature of the skin surrounding the wound. In other arrangements, the wound healing factor includes one or more of chemical levels, pH, fibrium, albumin, sodium salts, calcium, red blood cells, white blood cells, bacterial fauna, ions, cations, charge levels, voltage gradients, and tissue impedance or tissue resistance in the wound.

The control module may be configured to apply any suitable therapy to a wound. In one suitable approach, the control module may adjust the voltage potential to maintain a constant current density at the first electrode. In another suitable approach, the control module may adjust the voltage potential to cause a selected concentration of peroxide to be present in the wound bed. In yet another suitable approach, the control module may adjust the voltage potential to limit production of proteoglycans in the wound. In still another suitable approach, the control module may change an application of a first therapy to the wound to an application of a second therapy to the wound. The control module may be configured to store multiple therapies and to apply a selected one of the therapies to the wound.

The control module may also be configured to detect an infection in the wound. The control module may detect the infection through measurements from the feedback sensor. When an infection is detected, the control module may trigger an alarm to alert a health care practitioner to the infection or it may trigger the delivery of an additional electrical therapy parameter.

The control module may also be configured to turn on the voltage potential for a predetermined period of time and to turn off the voltage potential for a predetermined period of time, resulting in duty cycles for the application of a therapy. In another approach, the control module may alternate a selected electrode from being designated a cathode to being designated an anode. For example, in a system including a first electrode that is an anode and a second electrode that is a cathode, the control module may alternate the applied voltage to cause the first electrode to become the cathode and the second electrode to become the anode.

The control module may include a storage device such as memory and may be configured to store the feedback sensor output in the storage device. The control module may be configured to export the feedback sensor output from the storage device to an external device for analysis or for transmission to another input device via a wireless method such as a “Bluetooth” system.

The control module may also include a display. The control module may display, for example, the feedback sensor output on the display. In addition, the control module may have one or more settings, and may display the settings on the display.

The electrode system for applying a therapy to a wound may include a timer. The timer may be configured to indicate a time associated with the application of a therapy. For example, the timer may indicate the length of time the therapy has been applied to the wound. In another suitable approach, the timer may indicate the time remaining until the end of the therapy. In another yet suitable approach, the timer may control the polarity and/or the level of the voltage gradient applied.

In another embodiment of the present invention, the electrode system for applying a therapy to a wound may include multiple center electrodes. The electrode system may include a remote electrode that is configured to attach to the skin surrounding the wound. The control module is coupled to the center electrodes and the remote electrodes. The control module is configured to apply a voltage potential across the remote electrode and one or more of the center electrodes.

In another embodiment of the present invention, the electrode system may be provided with a control module implemented on a flexible circuit which is capable of conforming to the shape of the wound or the skin to which it is applied. The electrode system may also include a flexible support structure, and first and second electrodes, both coupled to the support structure. The first electrode may be applied to the wound, and the second electrode may be applied to skin surrounding the wound. The control module is coupled to the flexible support structure, and is configured to apply a voltage potential across the first electrode and the second electrode.

In accordance with another aspect of the present invention, components of the electrode system may be coupled together with connectors that can be selectively coupled to each other. For example, the connectors may allow certain electrodes to be coupled with only a subset of available control modules.

In a further aspect of the present invention, a medical kit may be provided that includes components for use in applying a therapy to a wound. The kit may include multiple control modules and multiple electrodes. The control modules may have a plurality of selected strengths and stored therapies and the electrodes may be of a plurality of sizes, shapes, and configurations.

### BRIEF DESCRIPTION OF THE DRAWINGS

The foregoing and other objects and advantages of the invention will be appreciated more fully from the following further description thereof, with reference to the accompanying drawings.

FIG. 1 is a functional block diagram of an electrode system in accordance with the present invention.

FIG. 2 is a flow diagram of a method implemented by an electrode system for applying a therapy to a wound in accordance with the present invention.

FIG. 3 is a top view of an illustrative electrode system applied to a wound in accordance with the present invention.

FIG. 4 is a top view of an illustrative electrode system in accordance with the present invention.

FIG. 5 depicts an exemplary set of electrode-power supply connections in accordance with a first embodiment of the present invention.

FIG. 6 depicts an exemplary set of electrode-power supply connections in accordance with a second embodiment of the present invention.

### DETAILED DESCRIPTION OF THE PREFERRED EMBODIMENTS

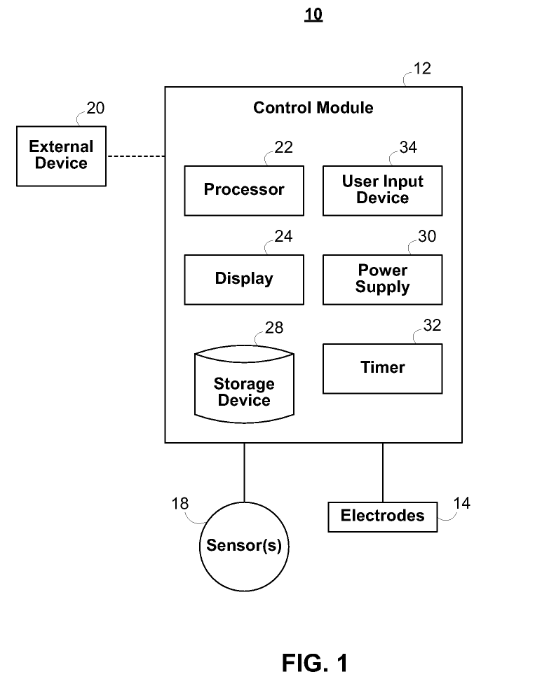

To provide an overall understanding of the invention, certain illustrative embodiments will now be described with reference to FIGS. 1-6. It will be understood by one of ordinary skill in the art that the systems, methods, and devices shown and described herein can be adapted and modified for other suitable applications and that such other additions and modifications will not depart from the scope hereof.

FIG. 1 is a functional block diagram of an electrode system 10 including a control module 12, electrodes 14, one or more feedback sensors 18, and an external device 20. Control module 12 is configured to apply a therapy to a wound through electrodes 14. Control module 12 may be constructed using any suitable hardware and/or software to implement the therapies described. In one example, the control module 12 may be programmed by a FLASH boot loader to custom configure delivery schemes. In another example, control module 12 may be a low current microcontroller (e.g. MSP430F149), and may provide a “SMART” drive of current. According to the illustrative arrangement, control module 12 includes a processor 22, a display 24, a storage device 28, a power supply 30, a timer 32, and a user input device 34. According to alternative arrangements, control module 12 may not include all of the depicted components. For example, control module 12 may not include display 24, storage device 28, timer 32, or user input device 34. In the simplest embodiment, control module 12 may be a battery.

Processor 22 of control module 12 may be any suitable type of processor or embedded controller, including, for example, a microprocessor/microcontroller, a digital signal processor, a digital light processor, a Complex Programmable Logic Device (CPLD), a Field Programmable Logic Array (FPGA), and a Very Large Scale Integration Application Specific Integrated Circuit (VLSI ASIC). The functionality of processor 22 may be implemented in software, using programming languages known in the art, hardware, application specific integrated circuits, programmable logic arrays, firmware, or a combination of the above. Processor 22 may be coupled to display 24, storage device 28, power supply 30, timer 32, user input device 34, electrodes 14, one or more sensors 18, and external device 20. Processor 22 may process data inputs to control module 12, control the components of control module 12, and apply or adjust the therapy applied to electrodes 14.

Display 24 of control module 12 may be any suitable type of display, including, for example, an LCD screen, an LED screen, or a touch screen. Display 24 may display information about the current therapy such as, for example, the strength of the therapy, the type of therapy, and the duration of time the therapy has been applied. If display 24 is a touch screen, the screen may be configured to display selectable options. The selectable options may allow an operator to change the settings of control module 12. For example, the selectable options may allow an operator to turn on or off the device, select a particular therapy to be applied, set an amount of time to apply the therapy, or select or change any other setting.

In addition to the touch screen embodiment of display 24, or as an alternative, control module 12 may include a separate user input device 34. User input device 34 may be any suitable input device or devices, including buttons, scrolling wheels as are commonly found on PDA's, a keypad, switches, and an interactive display such as a touchpad, which may allow the user to select elements on the display. User input device 34 alone, or in combination with display 24, may allow an operator to access and interact with all of the features and functionality of control module 12.

Control module 12 may include storage device 28, which may be configured to store information about different therapies that can be applied. Storage device 28 may be any suitable storage device, including, for example, memory such as Random Access Memory (RAM), flash memory, and cache memory, one or more hard disk drives, or any suitable combination thereof. Storage device 28 may store information used by processor 22 to implement one or more therapies, including varying the voltage applied to electrodes 14 based on measurements from sensor or sensors 18, or varying the current that is driven through the circuit in real time. Storage device 28 may also store information used by processor 22 to allow a user to choose a particular therapy for application and also to adjust other settings of control module 12. Storage device 29 may also be capable of being programmed on a custom basis by research clinicians. For example, information stored in storage device 28 may be used by processor 22 to display available therapies to an operator on display 24. Storage device 28 may also be used to store information about previously applied therapies. For example, storage device 28 may store sensor measurements outputted from feedback sensor or sensors 18, the length of time therapies were applied, and which particular therapies were applied. Data stored in the storage device 28 may be input to the processor 22, which may use the data to optimize the selection and/or delivery of therapy. For example, the stored data may be used to select the therapy type, or it may be used as input parameters for optimization of a selected therapy.

Control module 12 may also includes a power supply 30, which powers the various components of control module 12 and may also be used by processor 22 to generate a voltage that is applied to electrodes 14. In one embodiment, power supply 30 is a battery. In this embodiment, power supply 30 may be any suitable battery including, for example, an alkaline, nickel cadmium, lithium, lithium polymer, zinc carbon, zinc chloride, zinc air, or silver oxide battery. The power supply 30 may be a rechargeable battery. In addition to a battery, or an alternative, power supply 30 may include photovoltaic cells. One or more photovoltaic cell may be placed on the back of control module 12 and provide power to the control module when exposed to visible light.

In an alternative embodiment, power supply 30 may be separate from control module 12. In such an embodiment, power supply 30 may provide power to control module 12 using any suitable connection, such as a hard-wired connection. Power supply 30 may, for example, be a battery or a DC power supply.

In an alternative arrangement, the power supply 30 is inductively coupled to the control module 12 via a corresponding element (e.g., induction coils) and without wires or other physical connection means. In this arrangement, the control module 12 may use the power from the power supply 30 to provide power to the processor 22 and the electrodes 14.

Control module 12 may include a power level gauge coupled to power supply 30, which indicates the battery level or power remaining in the power supply. In one suitable approach, the power level gauge provides a visual indication of the battery level. The visual indication may be in the form of bars to indicate the remaining battery power. In another example, colors may be used to indicate the battery level (e.g. green may indicate that the battery is good, yellow may indicate the battery level is getting low, and red may indicate that the battery, or control module 12, should be replaced). The power level gauge may be incorporated into display 24, or may be a separate component. If the power level gauge is a separate component, the information may be displayed using LEDs, LCDs, electroluminescence, or any other suitable technology.

In another arrangement, the power level gauge may provide an audible indication of the battery level. For example, an alarm may be sounded or a voice indication may be played. The power level gauge may be configured to provide a continuous indication of battery level. Alternatively, the battery level gauge may be configured to provide an indication of battery level only when the battery level becomes low or reaches any other predetermined level or levels. In another suitable approach, the power level gauge provides battery level information on demand or in response to a user query. For example, control module 12 may include a button that allows a user to activate the power level gauge.

Control module 12 may also include a timer 32. Timer 32 may be configured to record the duration of time a therapy has been applied. For example, timer 32 may record the duration of time that a voltage or current has been applied to electrodes 14. Timer 32 may be coupled to the storage device 28 and store duration data in the storage device. Timer 32 may be implemented using an embedded microcontroller running logic or application specific software or firmware. Time 32 may be a separate component of control module 12 or its functionality may be incorporated into processor 22. In one suitable approach, timer 32 may measure the duration of time an electrode system or dressing has been applied to a wound or the skin. Timer 32 may be stopped when a therapy is turned off, and be started when a therapy is turned on, thus measuring the cumulative time that a therapy has been applied to a wound. In another suitable approach, timer 32 may measure the duration of application of a particular therapy, for example the most recent therapy. According to another suitable configuration, a user may set timer 32 to specify the amount of time a therapy is to be applied to a wound. In such a configuration, for example, electrode system 10 may be provided without feedback sensor or sensors 18.

In addition, control module 12 may include a visual or audio indicator (not shown) to allow an operator to determine whether or how well electrode system 10 is functioning. In one example, the indicator may indicate whether control module 12 is currently applying a voltage to the electrodes 14. The visual indicator may be a light emitting diode (LED), a series of LEDs, a basic current meter, or any other suitable visual indicator. The audio indicator may be an alarm or a voice clip.

According to the illustrative arrangement of FIG. 1, control module 12 is coupled to one or more feedback sensors 18. Each feedback sensor 18 is configured to detect one or more factors that affect wound growth or other treatment factors, and to provide an output to control module 12. For example, processor 22 may receive and process the sensor output. Feedback sensor 18 may be any suitable type of sensor, including, for example, a reactive sensor, an electrochemical sensor, a biosensor, a physical property sensor, a temperature sensor, a sorption sensor, a pH sensor, a voltage sensor, a current sensor, and any suitable combination thereof.

Feedback sensor 18 may be configured to detect any suitable factor or factors that affect the treatment of skin or wound growth, including, for example, the natural current of injury of the wound, the amount of peroxide being generated by an electrode placed in the wound or the amount of peroxide present in the wound bed, the temperature of the wound, and the temperature of the skin surrounding the wound. Other factors sensor 18 may be configured to detect include chemical levels, the amount of oxygen, the amount of carbon dioxide, pH, fibrium, albumin, sodium salts, calcium, red blood cells, white blood cells, bacterial fauna, ions, and cations in the wound. In various arrangements, electrode system 10 may include multiple sensors 18. Sensors 18 may be placed in any suitable location on the patient, including on the treated part of the skin, in the center of a wound, on an edge of the wound, or on healthy skin surrounding the wound.

In addition, sensors 18 may be configured to examine the surface of the electrodes to observe changes over time to determine the chemistry of what is occurring in the wound bed. The sensors 18 may be configured to detect the liberation of selected growth factors by the wound or surrounding tissue, the liberation of selected ionic species by the wound or surrounding tissue, or the liberation of selected biological chemicals or compounds that relate to the wound or surrounding tissue.

Electrode system 10 may include two or more electrodes 14. For treatment of a wound, electrode system 10 may include two electrodes, with a first electrode for positioning on the wound (e.g., at the center of the wound), and a second electrode for position on the skin surrounding a portion or all of the wound. In other arrangements, multiple electrodes may be placed in the wound and/or multiple electrodes may be placed around the wound.

Electrodes 14 may be thin metal, metallic paint or pigment deposition, metallic foil, conductive hydrogels, or any other suitable conductive material. Hydrogels are generally clear, viscous gels that protect the wound from dessicating. In one suitable approach, conductive hydrogels may be used as the material for electrodes 14 because of their permeability to oxygen and ability to retain water. Both oxygen and a humid environment are required for the cells in a wound to be viable. In addition, hydrogels can be easily cast into any shape and size. Various types of conductive hydrogels may be employed, including cellulose, gelatin, polyacrylamide, polymethacrylamide, poly(ethylene-co-vinyl acetate), poly(N-vinyl pyrrolidone), poly(vinyl alcohol), HEMA, HEEMA, HDEEMA, MEMA, MEEMA, MDEEMA, EGDMA, mathacrylic acid based materials, and siliconized hydrogels. PVA-based hydrogels are inexpensive and easy to form. The conductivity of such hydrogels can be changed by varying the salt concentration within the hydrogels. By increasing the salt concentration within a hydrogel, the conductivity of the hydrogel increases.

Control module 12 may be connected to electrodes 14 in any suitable manner. For example, control module 12 may be electrically coupled to the electrodes using any suitable conductive material, such as copper wire. In another example, control module 12 may be inductively or capacitively coupled to electrodes 14, such that at least a portion of the coupling is made with a wireless connection. Additionally, the connection between control module 12 and the electrodes 14 may be permanent, it may be removable, or it may be modular. A removable or modular connection may allow a single control module to be used to power multiple different electrodes over time. A removable connection may be removable from both control module 12 and electrodes 14, it may be removable only from control module 12, or it may be removable only from one or more of electrodes 14. The connection may be manufactured in any suitable length, for example, about 0.5 cm, about 1 cm, about 2 cm, about 3 cm, about 4 cm, about 5 cm, or greater than about 5 cm.

In one arrangement, electrode system 10 includes an external device 20, which may be coupled to control module 12. External device 20 may, for example, be a dedicated device for interfacing with control module 12 or may be computer system with the appropriate software and hardware for interfacing with control module 12. External device 20 may be used to program therapies in control module 12. Additionally, external device 20 may receive data outputted by control module 12 for storage or analysis. In one suitable approach, control module 12 may be configured to export data from feedback sensor or sensors 18 to the external device 20. The data on external device 20 may be analyzed and waveform optimization may be performed. A health care professional may, for example, analyze specific wound data over time (e.g. current of injury, temperature, growth factors, pH, and peroxide levels), and use the data to determine customized treatment therapies and/or to track the healing progress of the wound.

The data may be outputted to external device 20 through a wireless connection (e.g., using Bluetooth technology) from control module 12, or through a standard wired connection, such as a cable and a serial port. A cradle may be provided that interfaces with control module 12 and uploads the data to external device 20. The cradle may also be used to charge control module 12. Alternatively, the data may be outputted to device 20 though a removable storage device, such as a memory card.

As discussed above, control module 12 may be configured to provide many different types of therapies to the skin or a wound by applying a voltage potential across at least two of electrodes 14. Generally, control module 12 may be configured to provide a constant or varying voltage, a constant or varying current, or any other suitable electrical output to electrodes 14. Thus, the current density at the interface of electrodes 14 and the skin and wound may therefore be constant or time varying. When varying the voltage or current, control module 12 may cause electrodes 14 to change polarities at a constant or at a time varying frequency. In one suitable approach, control module 12 may be configured to pulse electrodes 14. In another suitable approach, the control module 12 may provide voltage or current to electrodes 14 in various stimulation wavetrains and signals, such as modulated AC, and spark gap waveforms. This may be implemented using processor 22 or using a waveform generator integrated circuit and an analog multiplier integrated circuit coupled to power supply 30 for generation of the various stimulation wavetrains and signals.

Control module 12 may apply therapies using a closed loop system or an open loop system. In the open loop system, control module 12 applies therapies without the use of feedback from one or more feedback sensors 18. In the closed loop system, control module 12 adjusts the therapies being applied based on the output from sensor or sensors 18. Control module 12 may adjust the therapy continuously based on the output from the at least one sensor (e.g., to keep a sensor measurement at a particular value), or may vary the therapy when one or more sensor measurements reach predetermined set point or points. In both the open and closed loop applications of therapies, control module 12 may allow an operator to adjust the voltage potential and polarity being applied to electrodes 14. For example, the operator may increase or decrease the gain of the applied voltage potential. The operator may adjust the gain using user input device 34 or external device 20.

Control module 12 may limit the maximum voltage potential that is applied across electrodes 14. For example, in one suitable approach, control module 12 may limit the maximum voltage to about 9 V. However, control module 12 may also be configured to apply voltage greater than 9 V. The appropriate voltage can vary depending on, for example, the size of electrodes 14, the location where the electrodes are applies, and the desired treatment desired. For example, a lower voltage may be used when the electrodes are smaller. Smaller electrodes have smaller surface areas and thus the current density would increase for the same voltage. High current density may be detrimental to the skin or the healing of a wound. In one suitable approach, control module 12 may limit the current density that is applied to the skin or wound to be less than about 10,000 μA/cm². However, in other suitable approaches, the applied current density may be greater than 10,000 μA/cm².

The operator of electrode system 10 may select the appropriate size and shape of electrodes 14 for the particular application. When electrode system 10 is used to apply a therapy to a wound, one of electrodes 14 may be placed on the wound. This electrode will be referred to as the center electrode. The center electrode may be smaller than the wound, the same size as the wound, or even larger than the wound. The size and exact placement of the center electrode may vary based on the type of wound, the size of the wound, the shape of the wound, and the location of the wound. Typically, the center electrode should be placed in the approximate center of the wound. Another one of electrodes 14 should be placed at least partially on the skin surrounding the wound. This electrode will be referred to as the remote electrode. In one preferred approach, the remote electrode is configured to completely surround the wound.

In one suitable embodiment, sensor 18 may be configured to detect a concentration of peroxide in the wound bed or the amount of peroxide being generated. Electrode system 10 may produce peroxide at the electrode that functions as the cathode. The cathode electrode is at a negative electrical potential relative to a reference or anode electrode. The production of peroxide has a bactericidal or bacteriostatic effect on the wound. However, high concentrations of peroxide may have a detrimental effect on the wound. Accordingly, one suitable therapy that may be applied by electrode system 10 to a wound is to maintain a selected concentration of peroxide in the wound bed. Based on measurements from sensor 18, control module 12 may increase or decrease the voltage applied to electrodes 14, thereby adjusting the amount of peroxide generated in the wound as described below.

Hydrogen peroxide is produced at an electrode through the electrochemical reduction of oxygen. According to one approach, the electrochemical reduction of oxygen requires that the noble metal (the cathode) be at a negative electrical potential relative to a reference electrode. In a laboratory environment, for example, electrodes of platinum (Pt) or gold (Au) may require from ˜0.6 to 0.8 V negative potential, or polarization, relative to a calomel or a silver/silver chloride (Ag/AgCl) reference electrode in potassium chloride of approximately pH 7 for the reaction to occur. Electrode pairs with a voltage potential difference of approximately 0.6-0.8 V may produce a current proportional to the concentration of oxygen in the system as the oxygen is decomposed. Greater voltage potential differences may lead to rapid increases in the current due to additional reactions, mainly the reduction of water to hydrogen.

The laboratory observations of the type discussed led others to the development of an oxygen sensing electrode based on Pt and Ag/AgCl. The reduction/oxidation (redox) reactions for the “Clark”—type of electrode pairs (also known as Clark oxygen sensing polarographic electrode reactions) are:

Cathodic reaction: O₂+2H₂O+2e−→H₂O₂+2OH⁻

H₂O₂+2e⁻→2OH⁻

Anodic reaction: Ag+Cl⁻→AgCl+2e⁻

Overall reaction: 2Ag+O₂+2H₂O+2Cl⁻→2AgCl+4OH⁻

As seen in the equations above, the generation and consumption of hydrogen peroxide (H₂O₂) and the generation of base (OH⁻) are involved in this chemistry. In the Clark type systems both H₂O₂ and OH⁻ have been noted. Appreciable levels of H₂O₂, which is an intermediate in the redox system, have been attributed to diffusion of H₂O₂ away from the electrode surface, preventing further reduction to OH⁻. An additional factor is the condition of the electrode surface which will influence the rate of the reactions involved. There are at least two alternative pathways in the above described electrode system: one with no H₂O₂ buildup and one with H₂O₂ buildup. An example pathway that does not include H₂O₂ buildup is:

O₂ (Bulk)→O₂ (Surface)+2e⁻→H₂O₂+2e⁻→H₂O

An example pathway that does cause H₂O₂ buildup is:

O₂ (Bulk)→O₂ (Surface)+2e→H₂O₂+[DIFFUSION]→H₂O₂

As discussed above, the circuitry employed in the application of electrical stimulation may produce a limited current at a controlled voltage. In addition to the electrode systems of the present invention, other exemplary electro-active therapy systems include, for example, the POSIFECT device, and similar devices, such as those described in commonly assigned U.S. Pat. No. 6,631,294, the entire contents of which are hereby incorporated by reference. POSIFECT is a trademark owned by Biofisica LLC. Experiments conducted using the POSIFECT device have demonstrated that when operated at 6 Volt (50 mA limited current by design), the electrodes of that system are capable of producing H₂O₂ in isotonic borate buffer at levels that range from approximately 3 parts per million (ppm) after 12 hrs, approximately 30 ppm after 27 hrs, and approximately 100 ppm after 36 hrs. Additionally, when the electrodes were operated at 6 V with no limit on the current, bubbles were observed at the cathode. Bubble formation at the cathode is consistent with the reduction of H₂O to hydrogen (H₂). It should be noted that additional electrolytic reactions are possible. For example, when sodium ion (Na+) is present, reduction to sodium metal)(Na⁰) is possible. Na⁰ spontaneously reacts with H₂O and generate additional H₂.

The experiments noted above indicate that the systems employed in electrical stimulation therapy may produce chemical species. The production of the chemicals may be time dependent and it may be spatially dependent, as described in further detail below. The production of certain chemicals, such as hydrogen peroxide, may facilitate wound healing.

The production and concentration of H₂O₂ at electrodes 14 may be determined by diffusion rates and reaction kinetics at the surface of the cathode electrode. Similar factors may influence the rate of OH⁻. Thus, it may be useful to identify the buffer capacity when using electrode system 10. Materials and electrical profiles employed in electrode system 10 may be selected to optimize the production of H₂O₂ and enhance the wound healing response. Additionally, O₂ levels may be manipulated to produce more or less O₂ in regions of the wound. Electrode system 10 and/or packing used with it may employ buffers that lead to the production of chemicals that are beneficial to various stages of wound healing. For example, when proud flesh is present, oxidizing and bleaching agents may be beneficial and encourage wound healing.

There are several techniques that can be used to increase the rate of H₂O₂ production for a given voltage potential. One technique is to increase the surface area of the cathode electrode to increase the rate of H₂O₂ production. Another technique is to use activated carbon particles dispersed in, for example, a hydrogel. The particle characteristics, such as particle size, porosity, and loading, would impact the rate of H₂O₂ formation. The hydrogel may be used as the material for electrodes 14 or the hydrogel may be placed on electrodes 14 such that it attaches the electrodes to the wound or skin.

The production of H₂O₂ may be spatially distributed over the wound surface by using a selected cathode geometry. For example, a wire mesh cathode, in conjunction with a remote anode, as described above, may generate H₂O₂ over a large area, with the H₂O₂ diffusing to the spaces between wires of the wire mesh, as well as away from the edges of the mesh. In this example, the cathode may be sized to cover a substantially large portion of the wound. Similarly, a gauze material may be selected for the cathode. According to other examples, the cathode and anode electrodes may be constructed in an alternating fashion of concentric rings, in a grid-node arrangement, or any other suitable arrangement. The cathode to anode distances can be optimized for the selected spatial distribution of H₂O₂.

The rates of electrolysis may be controlled by adjusting the voltage potential applied across electrodes 14. The relatively high voltage potential range available in electrode system 10 allows for a large variety of possible reactions. The determination of which reactions are thermodynamically likely to occur may be established using the reduction and oxidation potentials of the chemical species. In addition, the kinetics of the reactions may initially be determined by the current that flows through the wound and skin. Different electrode geometries, as discussed above, may add a further degree of control by establishing different current densities from the applied voltage. The cathode-to-anode surface area ratio may be optimized to provide another degree of control to the system. These factors, in combination with choices of electrode permeability and other possible membranes or materials to control fluxes and diffusion of species, may be used to deliver benefits to the wound healing process in addition to those provided by the voltage gradient between the electrodes. For example, buffers and packings associated with electrode system 10 may be selected according to the stage of the wound in the healing process or according to the particular therapy being applied to the wound and skin. The buffers may include soaking solutions, such as isotonic saline or buffered isotonic saline. The packings may include sterile gauze, dried or semi-dried hydrogels, and alginates.

In another suitable therapy, electrode system 10 may be configured to adjust a therapy to compensate for the natural current of injury. The natural current of injury may be detected by a feedback sensor 18. If the measured natural current of injury is low or below a desired amount, then control module 12 may apply a voltage potential to increase the current of injury. The current of injury can be increased to a level where it should be if the wound was healing correctly. Alternatively, the natural current of injury can be increased greater than where it would be if the wound was healing correctly.

In another suitable therapy, electrode system 10 may be configured to adjust the voltage potential applied across as least two of electrodes 14 in order to limit the production of proteoglycans in the wound. Proteoglycans, such as Chondroitin Sulfate Proteoglycans (CSPGs) and other compounds, may prevent nerve tissue regeneration. Exemplary means of limiting proteoglycan production may include electrical, electronic, and chemical means.

In another suitable therapy, electrode system 10 may be configured to produce or release at least one of: silver (e.g. the anode or cathode electrodes may be plated with silver to cause specific salts of silver to be generated and diffused into the wound as a biocidal agent); charcoal, carbon, or other odor absorption means; drugs or other molecules.

In another suitable therapy, electrode system 10 may deliver drugs or other molecules to the local environment of a wound using iontophoretic techniques incorporated within the electrode system. For example, if a hydrogel is used in the electrode system, the hydrogel may be impregnated with the drugs or selected molecules. The electrode system may be configured for timed-release of the drugs or selected molecules. In various arrangement, the selected drugs and other molecules may include one or more of the following: angiogenesis-enhancing agents, inflammation-reduction agents, growth factors, antibacterial agents, antimicrobial agents, pain reduction agents, odor control agents, exudates control agents, bactericidal agents, and bacteriostatic agents.

During the application of a therapy, control module 12 may be configured to detect an infection in the wound or a systemic infection. Control module 12 may detect an infection based on measurements from sensor 18. Control module 12 may be programmed to recognize certain sensor measurements, such as high skin temperature, as an indication of infection. According to one embodiment, control module 12 includes an alarm, which is triggered upon detection of an infection. The alarm may be, for example, a light, a flashing light, or an audio signal. In addition to the alarm, or as an alternative, control module 12 may be configured to change or modify the therapy to help stop the infection. For example, control module 12 may increase the amount of peroxide in the wound bed. Alternatively, control module 12 may make other changes to the therapy when an infection is detected.

Control module 12 may also be configured to switch from the application of a first therapy to the application of a second therapy. Control module 12 may switch between types of therapies after a predetermined amount of time or based on measurements from sensor or sensors 18. In some embodiments, control module 12 may switch the therapy by changing the voltage potential applied to electrodes 14.

Control module 12 may be configured to continuously apply a therapy or may be configured to turn on a therapy for a predetermined period of time and then turn off the therapy for a predetermined period of time. In this manner, control module 12 may provide various duty cycles. For example, control module 12 may be configured to turn on a therapy for 12 hours and then turn it off for 6 hours, and repeat. This example is merely illustrative. Control module 12 may be configured to turn on the therapy for any selected number of seconds, minutes, hours, days, or weeks and then off for any selected number of seconds, minutes, hours, days, or weeks. According one approach, control module 12 includes a 555 timer and a field-effect transmitter (FET) switch, which are utilized to provide different “on” and “off” periods or duty cycles for therapy application. To minimize the size of control module 12, the 555 timer and FET switch may be implemented in integrated circuits, which may be utilized in die-bond form. Various threshold levels, and therefore various duty cycles, may be set by selecting suitable fuses, contacts, or breaks in contacts in the integrated circuits.

As discussed above, control module 12 causes at least a first electrode to have a first polarity and at least a second electrode to have an opposite polarity. Control module 12 may vary the polarity of the electrodes to elicit specific results. Exemplary effects on the wound from varying electrode polarity include production of growth factors, reduction of inflammatory response, growth of blood vessels, production of collagen, and formation of epithelial tissue at a selected time.

For example, the first electrode may be placed in the wound and designated as a cathode, which has an antibacterial effect on the wound and may help eliminate chronic inflammatory stimulus. A cathode may also stimulate fibroblast proliferation in the wound, since fibroblasts are positively charged. Fibroblast proliferation may increase the number of non-senescent cells. Additionally, keratinocytes may migrate toward a cathode, helping to stimulate re-epitheliazation. In another example. the first electrode is designated as an anode, which recruits fresh macrophages (since these have a negative charge) helping to decrease inflammation, normalize growth factor and cytokine profile, and decrease protease production. Switching the polarities of electrodes 14 may prevent tarnishing of the electrodes.

Control module 12 is configured to store a plurality of therapies that can be applied to a wound and skin. In one embodiment, each control module 12 may be configured to store all of the possible therapies that can be applied. In another suitable embodiment, control modules 12 may store only one or a subset of the therapies. Therefore, the electrode systems may be produced for a particular use or for a particular class of uses. For example, different electrode systems may be used by an operator used for each of the three different phases of wound healing. In another suitable example, different electrode systems may be used based on the size of the area to be treated. For example, a more powerful electrode system may be used on larger areas and a less powerful electrode system may be used on smaller areas. In another suitable example, different electrode systems may be used to obtain different desired results.

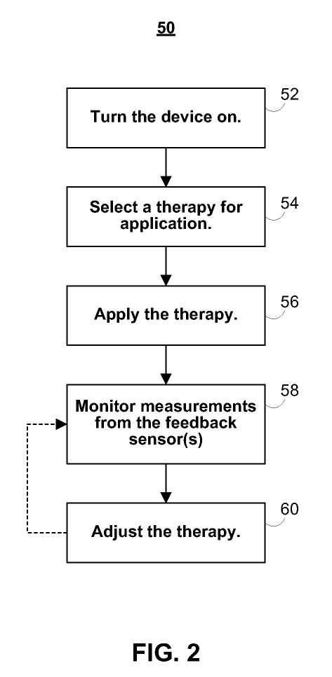

FIG. 2 is a flow chart of illustrative steps that may be involved in applying a therapy in accordance with the present invention. At step 52, an operator may turn on the device (e.g., electrode system 10). The operator may turn on the device by, for example, making an appropriate entry or selection on the device. This step may be performed before or after the device has been applied to the desired location. At step 54, the operator may select an appropriate therapy to be applied. For example, the operator may select a therapy designed to facilitate the healing of a wound during the proliferative phase. The operator may also select an amount of time that the therapy should be applied and may also set up a duty cycle for the therapy. Once electrode system 10 has been configured with the appropriate therapy, the therapy may be applied to the wound and/or skin at step 56. The therapy may be applied by having control module 12 apply a voltage or current to at least two electrodes, thereby causing a current to flow through the wound and/or skin. At least one feedback sensor, such as feedback sensor 18 of FIG. 1, may be positioned on a wound or the skin and provide an output regarding wound growth factors or treatment factors to the device. At step 58, the device monitors the measurement data from the feedback sensor or sensors. The measurement data may be recorded for later analysis. At step 60, the therapy being applied may be adjusted based on the data received from sensor or sensors. For example, control module 12 may increase or decrease the gain of the voltage or current being applied to electrodes 14 based on the received data. In another suitable example, control module 12 may change the type of therapy being applied based on the received data. As illustrated, the device may continue to monitor the feedback measurements and adjust the therapy accordingly.

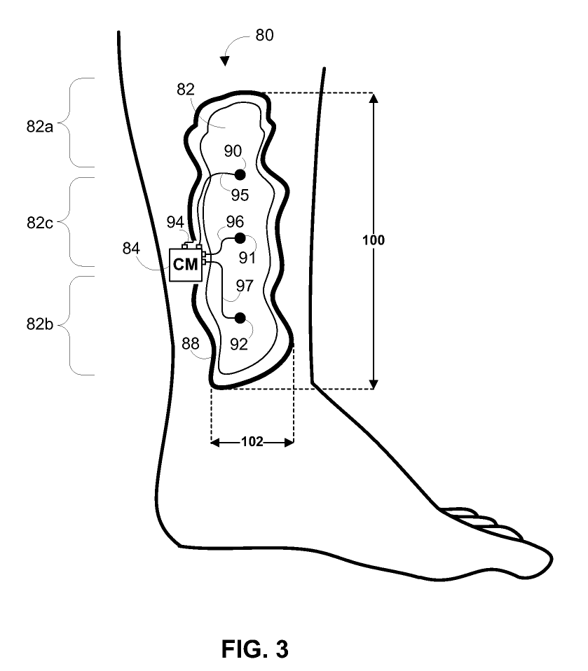

FIG. 3 is a top view of an illustrative electrode system 80 applied to a wound 82 in accordance with the present invention. The electrode system 80 includes a control module 84, a first electrode 88, and three center electrodes 90, 91, and 92. The system also includes four conductive leads 94, 95, 96 and 97, which connect electrodes 88, 90, 91 and 92, respectively, to control module 84.

The wound 82 as shown in FIG. 3 has a length 100 and a width 102. The length 100 is substantially greater than the width 102. Thus, three center electrodes 90, 91, and 92 may be placed in wound 82, substantially evenly spaced out along the length 100 of the wound 82. Using three center electrodes in this type of wound can provide a more even distribution of the generated current flow throughout the wound 82 than may be generated using only a single center electrode. If a single center electrode were placed in the center of the wound, the top 82a and bottom 82b ends of wound 82 may not receive as much current flow as the center section 82c of wound 82.

In one suitable approach, control module 84 may generate a voltage potential across first electrode 88 and all of center electrodes 90, 91, and 92. In this approach, center electrodes 90, 91 and 92 may act as cathodes, while the first electrode acts as an anode. According to another suitable approach, control module 84 may generate a voltage potential across first electrode 88 and one of center electrodes 90, 91, and 92. For example, control module 84 may apply the therapy using center electrode 90. After a predetermined period of time, control module 84 may switch from applying voltage to center electrode 90 and begin applying voltage to center electrode 91. Control module 84 may then switch from center electrode 91 to center electrode 92. Accordingly, control module 84 may sequentially apply voltage to each of center electrodes 90, 91, and 92 in any suitable order. According to another suitable approach, control module 84 may generate a voltage potential across first electrode 88 and two of the three center electrodes 90, 91, and 92. Control module 84 may then sequential apply voltage to different groups of two center electrodes.

The electrode system depicted in FIG. 3 is merely illustrative. Any suitable number of first and center electrodes may be used to obtain a desired current flow through the wound. For example, 2 center electrodes or 4 or more center electrodes may be used in accordance with the present invention. Preferably, an operator such as a health care professional will select the number, size and shape of the electrodes based on the size, shape, and condition of the wound.

In one implementation, electrode system 80 of FIG. 3 may include a top overlay layer (not shown), to which electrodes 88, 90, 91, and 92 are attached. Electrodes 88, 90, 91 and 92 may have an adhesive side for affixing electrode system 80 to wound 82, or a conductive adhesive may be attached to the underside of the electrodes 88, 90, and 92.

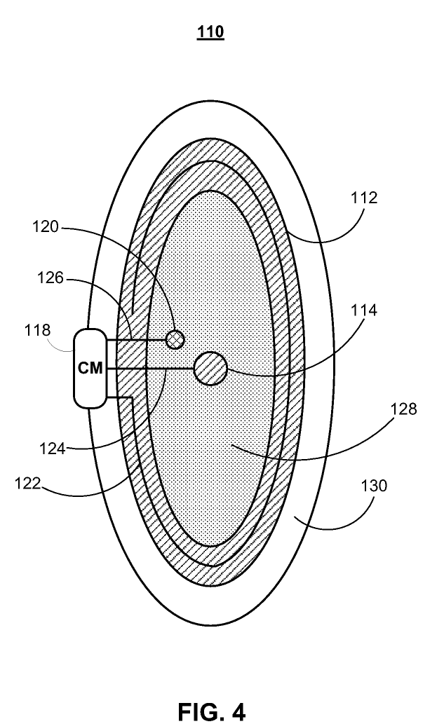

FIG. 4 is a top view of an illustrative electrode system 110, including electrodes 112 and 114, and control module 118 in accordance with the present invention. According to the illustrative arrangement, electrode system 110 includes a feedback sensor 120. Conductive leads 122 and 124 connect electrodes 112 and 114 to control module 118. Additionally, lead 126 connects feedback sensor 120 to control module 118.

According to one aspect of the present invention, control module 118 may be flexible, such that it conforms to the shape of the area on the body to which it is affixed. Control module 118 may be constructed using soft or flexible components. For example, the housing of control module 118 may be constructed of a soft or flexible material, and the circuitry of control module 118 may be a flexible circuit. The flexible circuit may be any suitable flexible circuit, including a single-layer circuit or a double layer circuit. The flexible circuit may be constructed of any suitable materials, including, for example, polyimide, copper, and photoimageable dry film. The flexible circuits may also include an adhesive, which may be any suitable adhesive, including epoxy, modified epoxy, phenolic butyral, acrylics, or modified acrylics. According to one approach, a flexible circuit may be constructed of substantially the same materials used for rigid circuit boards, with the exception that the substrate is flexible, not rigid. For example, a thin flexible plastic of metal foil may be used as the substrate. A flexible control module 118 may be useful if the area of the body to which it is applied is not flat. A flexible control module 118 may also be useful if pressure is used when electrode system 110 is applied to the body. Pressure may be applied, for example, if wrappings are used over electrode system 110.

Electrode system 110 as shown in FIG. 4 may be supplied as a prepackaged system that may be applied directly to a wound, such as wound 82 of FIG. 3, in a manner similar to the application of a typical dressing or bandage. Electrode system 110 may be supplied in a range of sizes and shapes. Electrode system 110 includes an insulative layer 128 and a top overlay layer 130. Electrode system 110 may also have an adhesive backing, and a layer to protect the adhesive backing prior to affixing electrode system 110 to a wound site.

Electrode systems may be supplied as a single unit such as electrode system 110 of FIG. 4 or may be supplied as component pieces such as in electrode system 80 of FIG. 3. In one suitable arrangement, the control module may be supplied separately from the electrodes. This arrangement allows an operator to select the appropriate control module and electrodes for a particular application. The electrodes may be configured to be attached to the control module using any suitable connection.

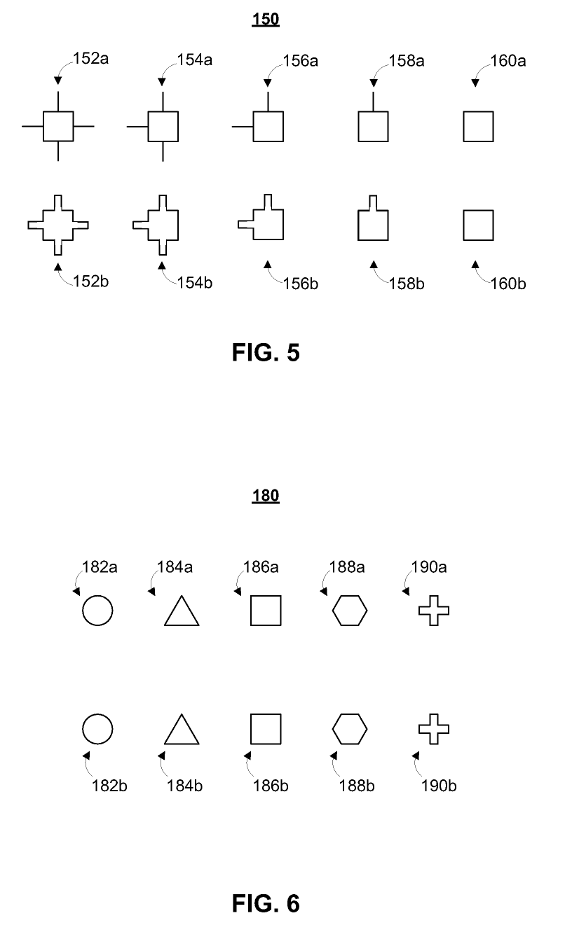

FIGS. 5 and 6 depict cross-sectional views of exemplary sets 150 and 180 of connectors in accordance with the present invention. Set 150 of FIG. 5 includes five male connector ends 152a, 154a, 156a, 158a, and 160a and five female connection ports 152b, 154b, 156b, 158b, and 160b. According to one arrangement, the male connector ends 152a, 154a, 156a, 158a, and 160a, may extend from connectors coupled to different electrodes, such as electrodes 14 of FIG. 1, while the female connection ports 152b, 154b, 156b, 158b, and 160b may be located on control modules, such as control module 12 of FIG. 1. As illustrated, the connector end 152a is configured for insertion only into connection port 152b, connector end 154a is configured for insertion only into connection ports 154b or 152b, connector end 156a is configured for insertion only into connection ports 156b, 154b, or 152b, connector end 158a is configured for insertion only into connection ports 158b, 156b, 154b or 152b, and connector end 160a is configured only for insertion into connection ports 160b, 158b, 156b, 154b or 152b. Thus, for example, connector end 156a can be inserted into three of the connection ports (i.e., connection ports 152b, 154b, and 156b), but can not be inserted into two of the connection ports (i.e., connection ports 158b and 160b).

The different connector ends may be used on different types or sizes of electrodes. For example, connector end 152a may be used on small electrodes and connector end 160a may be used on large electrodes. Similarly, the different connector ports may be used on different types and strengths of control modules. For example, connector port 152b may be used on a control module that has a small power supply and connector port 160b may be used on a control module that has a large power supply. Accordingly, in this example, the small electrodes can only be coupled to the control modules that have small power supplies. However, the large electrodes can be coupled to any of the control modules. This is merely illustrative. The electrodes may be supplied in more than two sizes and the control modules may be supplied in more than two strengths. Connector set 150, therefore, may be used to provide a safety mechanism, preventing a health care professional from inadvertently choosing a control module that is too strong for a selected electrode.

FIG. 6 depicts a cross-sectional view of an alternative set 180 of connectors. Set 180 of FIG. 6 includes five male connector ends 182a, 184a, 186a, 188a, and 190a, and five female connection ports 182b, 184b, 186b, 188b, and 190b. According to one arrangement, male connector ends 182a, 184a, 186a, 188a, and 160a extend from connectors coupled to different electrodes, while female connection ports 182b, 184b, 186b, 188b, and 160b may be located on different control modules. As illustrated, connector end 182a is configured for insertion into connection port 182b, connector end 184a is configured for insertion into connection port 184b, connector end 186a is configured for insertion into connection port 186b, connector end 188a is configured for insertion into connection port 188b, and connector end 190a is configured for insertion into connection port 190b.

The connector ends illustrated in FIG. 6, similar to the connection ends shown in FIG. 5, may be used on different types or sizes of electrodes. Accordingly, connector set 150 may be used to provide a safety mechanism, preventing a health care professional from inadvertently connecting electrodes to the wrong control module. For example, connectors 180 may be used such that electrodes of a particular size will only be able to couple to control modules of a matching power strength.

Connector sets 150 and 180 of FIGS. 5 and 6, respectively, can also be used to couple other components of electrode systems together. For example, these connector sets can be used to couple sensors to control modules. For example, different types of control modules may be supplied to work with different types of sensors. Therefore, connector sets 150 and 180 can be used to ensure that the correct sensor or sensors are used with the correct control modules. This is merely illustrative. Any other suitable combination of components may be coupled using connector sets 150 and 180. In an alternative example, “keyed” connector sets may identify their configuration to the control module(s) by the shorting of different pin pairs, thereby allowing a single control module to “adapt” its algorithms for communicating with various sensors.

According to another aspect of the present invention, medical kits may be supplied to health care practitioners that include a set of control modules and a set of electrodes. The control modules included in a medical kit may be of different types. For example, control modules of different strengths, control modules that apply different therapies, and/or control modules that use different sensors may be supplied as part of the medical kits. Similarly, the electrodes included in a medical kit may be of different types. For example, electrodes of different sizes, shapes, and configurations may be supplied as part of a medical kit. In addition, a set of sensors may be included as part of the medical kit. The sensors may be incorporated into the electrodes or they can be supplied as separate components. The sensors included as part of the medical kit may all be the same type of sensor or they may be different types of sensors. The control modules, electrodes, and sensors may use connectors such as those shown in connector sets 150 and 180 of FIGS. 5 and 6 to ensure that the sensors and electrodes are not incorrectly coupled to the control modules.

The medical kits may also include all of the necessary components for applying a complete regimen of therapies to a wound or the skin. Such a kit may include instructions and advice for applying the therapies, tools useful for treatment preparation or wound debriedment such as gauze, scalpels, scissors, tape, wound exudates absorbers such as alginates, and gauze or wound odor absorbers such as charcoal. The kit may also include a diagnostic device such as external device 20 of FIG. 1, a customized multi-meter to measure current or voltage or other biosensors to measure, for example, the current of injury or the specific biochemistry of the wound. The kit may include any items commonly found in first-aid kits, such as surgical tape, alcohol swabs, latex gloves, rubber gloves, and bandages.

The foregoing is merely illustrative of the principles of this invention and various modifications can be made by those skilled in the art without departing from the scope and spirit of the invention. For example, the electrode systems and methods described herein may be used for applications other than wound healing such as scar reductions, wrinkle reductions, improved quality of tissue deposition, hair growth, and on the face and neck after, for example, dermal peeling following laser or chemical facial peels. In addition, the electrodes systems and methods may be used in veterinary applications. For example, the electrode systems and methods may be used to treat skin conditions on horses.

What is claimed is:

1. An electrode system for applying a therapy to a wound, comprising:
a first electrode that is configured at least in part to be applied to the wound;
a second electrode that is configured at least in part to be applied to one of skin surrounding the wound and an outer portion of the wound;
a control module coupled to the first electrode and the second electrode, wherein the control module is configured to apply a voltage potential across the first electrode and the second electrode; and
a feedback sensor coupled to the control module, wherein the control module is configured to adjust the applied voltage potential based on the output from the feedback sensor, thereby adjusting the therapy applied to the wound.

2. The system of claim 1, wherein the feedback sensor is configured to detect at least one factor that affects wound growth and provide an output to the control module.

3. The system of claim 2, wherein the at least one factor comprises at least one of a natural current of injury of the wound, an amount of peroxide being generated by the first electrode, a temperature of the wound, and a temperature of the skin surrounding the wound.

4. The system of claim 2, wherein the at least one factor comprises at least one of chemical levels, pH, fibrium, albumin, sodium salts, calcium, red blood cells, white blood cells, bacterial fauna, ions, and cations in the wound.

5. The system of claim 1, wherein the control module is configured to apply a current to the first electrode and the second electrode, thereby applying a voltage potential across the first electrode and the second electrode.

6. The system of claim 5, wherein, based on the output from the feedback sensor, the control module is configured to adjust a current applied to at least one of the first and second electrodes.

7. The system of claim 5, wherein, based on the output from the feedback sensor, the control module is configured to adjust the voltage potential to result in a selected current density at one of the first and second electrodes.

8. The system of claim 1, wherein the control module adjusts the voltage potential to maintain a constant current density at the first electrode.

9. The system of claim 1, wherein the control module adjusts the voltage potential to produce a selected amount of peroxide.

10. The system of claim 1, wherein the control module adjusts the voltage potential to limit production of proteoglycans in the wound.

11. The system of claim 1, wherein the control module adjusts the voltage potential to change an application of a first therapy to the wound to an application of a second therapy to the wound.

12. The system of claim 1, wherein the control module is configured to detect an infection in the wound.

13. The system of claim 8, wherein the control module comprises an alarm and wherein the control module triggers the alarm upon detection of an infection.

14. The system of claim 1, wherein the control module is configured to turn on the voltage potential for a predetermined period of time and to turn off the voltage potential for a predetermined period of time.

15. The system of claim 1, wherein the voltage potential applied by the control module causes the first electrode to have a first polarity and the second electrode to have an opposite polarity.

16. The system of claim 11, wherein the control module is configured to switch the polarities of the first and the second electrodes.

17. The system of claim 1, wherein the control module is configured to store a plurality of therapies and wherein the control module is configured to apply one of the plurality of therapies to the wound.

18. The system of claim 1, wherein the control module comprises a storage device and wherein the control module is configured to store the feedback sensor output in the storage device.

19. The system of claim 18, wherein the control module is configured to export the feedback sensor output from the storage device to an external device.

20. The system of claim 18, wherein the control module is configured to use data stored in the storage device to adjust the therapy applied to the wound.

21. The system of claim 1, wherein the control module comprises a display and wherein the control module is configured to display the feedback sensor output on the display.

22. The system of claim 1, wherein the control module comprises a display, wherein the control module has settings, and wherein the control module is configured to display the settings on the display.

23. The system of claim 1, further comprising a power supply coupled to the control module.

24. The system of claim 23, wherein the power supply is inductively coupled to the control module.

25. The system of claim 1, wherein the feedback sensor is a biosensor.

26. The system of claim 1, wherein the feedback sensor is configured to detect at least one of release of selected growth factors, release of selected ions, release of selected biological chemicals, and release of selected biological compounds by the wound

27. An electrode system for applying a therapy to a wound, comprising:
a first electrode that is configured at least in part to be applied to the wound;
a second electrode that is configured at least in part to be applied to skin surrounding the wound; and
a control module coupled to the first electrode and the second electrode, wherein the control module is configured to apply a voltage potential across the first electrode and the second electrode, thereby applying a therapy to the wound, wherein the control module comprises a timer and wherein the timer is configured to indicate the length of time the therapy has been applied to the wound.

28. An electrode system for applying a therapy to a wound, comprising:
a first electrode configured to surround at least a portion of the wound;
a plurality of center electrodes configured at least in part to be applied to the wound; and
a control module coupled to the first electrode and the plurality of center electrodes, wherein the control module is configured to apply a voltage potential across the first electrode and one of the plurality of center electrodes.

29. The system of claim 28, wherein the control module is configured to switch from applying a voltage potential across the first electrode and the one of the plurality of center electrodes to applying a voltage potential across the first electrode and another of the plurality of center electrodes.

30. An electrode system, comprising:
a flexible support structure;
a first electrode that is coupled to the support structure and is configured at least in part to be applied to the wound;
a second electrode that is coupled to the support structure and is configured at least in part to be applied to skin surrounding the wound; and
a control module coupled to the support structure, wherein the control module is configured to apply a voltage potential across the first electrode and the second electrode and wherein the control module comprises a flexible circuit that conforms to the shape of the wound or the skin to which it is applied.

31. A system of components for electrodes systems, comprising:
a plurality of types of control modules configured to apply a voltage potential across electrodes; and
a plurality of types of electrodes,
wherein one type of the electrodes is configured to couple to at least one type of the control modules, and wherein the one type of the electrodes is configured such that is can not couple to another type of the control modules.

32. A medical kit for use in applying a therapy, comprising:
a plurality of types of electrode systems that are included in the medical kit, wherein a first type of electrode systems is different than a second type of electrode systems.

33. The medical kit of claim 30, wherein the first type of electrode systems is more powerful than a second type of electrode systems.

34. The medical kit of claim 30, wherein the first type of electrode systems is configured for application to a larger wound and wherein the second type of electrode systems is configured for application to a smaller wound.
